(19) United States
(12) Patent Application Publication
(10) Pub. No.: US 2008/0069795 A1
(43) Pub. Date: Mar. 20, 2008

(54) Amelioration of Drug-Induced Toxicity

(75) Inventor: Hamid Rabb, Ellicott City, MD (US)

Correspondence Address: BANNER & WITCOFF, LTD., 1100 13th STREET, N.W., SUITE 1200, WASHINGTON, DC, 20005-4051, US

(73) Assignee: The John Hopkins University, Baltimore, MD (US)

(21) Appl. No.: 11/631,366

(22) PCT Filed: Jun. 29, 2005
(86) PCT No.: PCT/US05/22860

§ 371 Date: Sep. 17, 2007

Related U.S. Application Data
(60) Provisional application No. 60/583,731, filed on Jun. 29, 2004.

(51) Int. Cl. A61K 39/00; A61K 31/138; A61K 31/282; A61K 39/395; A61P 35/00; A61K 38/18; A61K 38/20

(52) U.S. Cl. 424/85.2; 424/130.1; 424/140.1; 424/141.1; 424/184.1; 514/12; 514/492; 514/648

(57) ABSTRACT

Kidney toxicity which is induced by cisplatin and other therapeutic and diagnostic agents, limits the effectiveness of the therapy or diagnosis. Modulation or depletion of T cells ameliorates the toxicity, permitting the use of cisplatin at levels and for durations which treat cancers more effectively. Modulation and depletion can be accomplished using antibodies for T cell surface antigens as well as using other molecules which effectively antagonize or down-regulate the cytokines and/or chemokines which T cells elaborate.

This application claims the benefit of provisional application Ser. No. 60/583,731 filed, Jun. 29, 2004, the disclosure of which is expressly incorporated herein.

This invention was made using funds from the United States government. According to the terms of grants NIDDK RO1 DK54770 and NHLBI SCCOR HL073944, the U.S. government retains certain rights in the invention.

### TECHNICAL FIELD OF THE INVENTION

This invention is related to the area of preventing and treating organ-toxic side effects of chemotherapy. In particular, it relates to preventing and treating to ameliorate nephrotoxicity due to a platinum-containing compound.

### BACKGROUND OF THE INVENTION

Cisplatin (cis-diamminedichloroplatinum II) is an effective chemotherapeutic agent widely used in the treatment of a variety of malignancies including head and neck, ovarian and testicular cancers. However, nephrotoxicity, the most common adverse effect, limits the use of this drug in many cancer patients. (1) Approximately 25-30% of patients developed renal dysfunction after a single dose of cisplatin. (2) The pathogenesis of cisplatin toxicity is attributed to the formation of reactive oxygen species, (3) caspase activation, (4) DNA damage, (5;6) and mitochondrial damage. (7) Apoptosis, necrosis and inflammation have also been recognized as important mechanisms of cisplatin nephrotoxicity in vivo and in vitro. (8;9)

Recent studies have shown that cisplatin upregulates the expression of tumor necrosis factor alpha (TNF-α) in mouse kidney, and the level of TNF-α correlates with the severity of renal injury (10;11). Furthermore, inhibiting TNF-α release or its activity by using an antagonist, its inactive analogue, salicylate, or by using specific mice with genetic defects in TNF-α receptor 2 (TNFR2) protected mice from cisplatin-induced kidney injury (12;13). Moreover, T cells have been shown to be important modulator of ischemia reperfusion injury (IRI) in the kidney, liver, lung and the intestine (14-17). There is a continuing need in the art to identify methods for decreasing side-effects of chemotherapeutic drugs so that their full potential can be realized.

### SUMMARY OF THE INVENTION

According to one embodiment of the invention a method to prevent platinum-containing compound-induced kidney toxicity in a patient is provided. T cells in the patient are depleted prior to a planned administration or concomitant with administration of a platinum-containing compound.

Another embodiment of the invention is a method to treat platinum-containing compound-induced kidney toxicity in a patient in need thereof. T cells in a patient that has been treated with a platinum-containing compound are depleted.

Another aspect of the invention is a method to prevent platinum-containing compound-induced kidney toxicity in a patient. T cell activity in a patient is modulated such that level of IFN-γ in the patient's peripheral blood is less than 50% of unmodulated level. The patient is scheduled for treatment with platinum-containing compound or is treated with a platinum-containing compound concomitantly.

Still another aspect of the invention is a method to treat platinum-containing compound-induced kidney toxicity in a patient. T cell activity in a patient is modulated such that level of IFN-γ in the patient's peripheral blood is less than 50% of unmodulated level. The patient has been treated with a platinum-containing compound.

Another embodiment of the invention is a kit for treating cancers. The kit comprises a platinum-containing compound and an agent selected from the group consisting of: of

IL-10, TGF-beta, CD152, CTLA-4-Ig, Tamoxifen, and TJU103. The platinum-containing compound and the agent are in a single divided or undivided container.

Yet another embodiment of the invention is a kit for treating cancers. The kit comprises a platinum-containing compound and an antibody selected from the group consisting of: anti-CD4, anti-CD8, anti-CD28, anti-CD3, anti-CD52, anti-ICOS receptor, anti-PD1, anti-CD154, and mAb Hyb-241. The platinum-containing compound and the agent are in a single divided or undivided container.

These and other embodiments which will be apparent to those of skill in the art upon reading the specification provide the art with kits and methods for therapy of cancers.

### BRIEF DESCRIPTION OF THE DRAWINGS

FIG. 1. Survival in cisplatin-treated wild type mice and nu/nu mice. All mice received a single dose of cisplatin (i.p., 40 mg/kg) and were followed up to 72 hrs. Compared to 58% of survival rate in wild type mice, nu/nu mice had 100% of survival rate at 72 hrs after cisplatin administration (n=12-14).

FIG. 2. Renal function in cisplatin treated wild type mice and nu/nu mice. Serum creatinine was measured before (0 hr) and at 24 hr, 48 hr and 72 hr after having received injection of cisplatin (40 mg/kg). Compared to wild type mice, nu/nu mice had significant reduced creatinine elevation at all time points. (*, P=0.01; **, P<0.05; ***, P<0.0001 vs. WT, n=5-7)

FIG. 3. Renal tubular injury scoring in cisplatin treated wild type mice and nu/nu mice. The degree of mice renal tubular injuries at 72 hr after cisplatin administration (40 mg/kg) in wild type mice and nu/nu mice were scored using an established method of semi-quantitative evaluation. Compared to wild type mice that developed extensive tubular injury with a high score, the nu/nu mice had significantly less tubular injury. *, P<0.0001)

FIG. 4. FACS analysis of mouse splenic CD3 positive T cells. Mice were injected with cisplatin (40 mg/kg) and sacrificed at 72 hr after injection. One group of nu/nu mice (n=5) received a T cell adoptive transfer three weeks before the cisplatin administration. Splenocytes were isolated from each mouse upon sacrifice and stained with FITC-conjugated anti mouse CD3 antibody and analyzed by FACS. Wild type mice had (7.7±0.3) % of splenic T cells; meanwhile, nu/nu mice alone had minimal T cells (0.4±0.1) % in their spleen. Three weeks after receiving an adoptive transfer of wild type T cells, The average population of splenic T cells in those nu/nu mice was reconstituted up to (2.4±0.4) %. (nu/nu vs, nu/nu+T cell, P=0.0005)

FIG. 5. Effect of T cells transfer on post cisplatin mice renal function. Serum creatinine was measured in the wild type mice, nu/nu mice alone and the nu/nu mice with T cell transfer 72 hrs after received a single dose of cisplatin (40 mg/kg). Compared to nu/nu mice alone, there was a significant rise in serum creatinine in the nu/nu mice with T cells transfer. (*, P<0.05 vs nu/nu mice alone; **, P=0.002; ***, P<0.0001 vs. wild type mice. n=3-5).

FIGS. 6A-H. Microphotographs representing mice renal tubular injuries 72 hrs post cisplatin. FIGS. 6A and 6B: Wild type mouse with saline; FIGS. 6C, 6D: wild type mice with cisplatin; FIGS. 6E, 6F: Nu/nu mouse alone with cisplatin; FIGS. 6G, 6H: Nu/nu mouse with T cells transfer and cisplatin. Compared to the severe tubular injury showed in wild type mice, nu/nu mice showed less injury. Transferring wild type T cells into nu/nu mice significantly restored renal tubular injury.

FIG. 7. Semi-quantitation of mice renal tubular injury. Tubular injury was defined as tubular epithelium Necrosis, Cell Loss, (Intratubular, IT) debris and (hyaline) Cast formation. Injury scoring was based on the percentage of affected tubules in a high powered field and graded as following: 0, none; 0.5, <10%; 1, 10-25%; 2, 26-50%; 3, 51-75%; 4, >75%. Wild type mice had very severe tubular injury in all categories, whereas nu/nu mice showed mild injury. T cell transfer significantly restored nu/nu mice renal tubular injuries (*, P<0.022; **, P<0.0001, n=3-5).

FIG. 8. Quantification of CD3 positive cells on wild type mice post cisplatin kidney sections. Mice kidney tissues were stained for infiltrated T cells with an anti CD3 antibody by immunohistochemical technique. The positive stained T cells were counted in at least 10 high powered fields in cortico-medulary zone by a pathologist and a nephrologist in blinded fashion. Compared to the saline controls, CD3 positive cells were significantly increased at as early as 1 hr post cisplatin and peaked at 12 hr, declined by 24 hr. (*, P<0.004; **, P<0.0002 vs. saline)

FIG. 9. Renal myeloperoxidase (MPO) activity in nu/nu mice and wild type mice all mice received either cisplatin (40 mg/kg) or equal volume of saline and were sacrificed at 72 hr after treatment. Both saline treated wild type mice and nu/nu mice had a comparable base line of MPO activity. Compared to their individual saline controls, both wild type mice and nu/nu mice had significant increase in renal MPO activity at 72 hr after cisplatin. However, this increase in nu/nu mice was significantly blunted when compared to wild type mice. (*, P<0.001 vs. wild type control mice, P<0.04 vs. nu/nu control mice; #, P<0.04 vs. wild type cisplatin mice)

FIG. 10. Survival comparison among cisplatin treated wild type, CD4−/− or CD8−/− mice. By 72 hrs after cisplatin treatment, compared to 50% of survival in wild type mice, CD4−/− mice had 100%, CD8−/− mice had 80% of much improved survival rate, indicating both CD4−/− mice and CD8−/− mice were protected from cisplatin induced mortality. (n=6-12 per group)

FIG. 11. Pro-inflammatory protein array in post cisplatin mice kidneys. Kidneys were harvested at 24 hr or 72 hr after cisplatin injection and a multiplex cytokine/chemokines protein array was performed by Bio-Rad multiplex techniques. Compared to the controls baseline, there was significant increase in IL-1β, KC and TNF-α in wild type cisplatin treated mice at 72 hr. However, at this time point, the nu/nu mice had a significantly reduced increase in these cytokines when compared to wild type cisplatin injected mice. No significant increase was found in IFN-γ at either 24 hr or 72 hr after cisplatin administration in both wild type and nu/nu mice. (*, P<0.002; **, P<0.01; ***, P<0.02 vs. WT 72 hr. n=3 in each group)

### DETAILED DESCRIPTION OF THE INVENTION

The inventor has developed methods for preventing and/or treating toxicity associated with platinum-containing compound chemotherapy. Platinum-containing compounds can cause, inter alia, kidney failure. The risk of this adverse outcome requires that the oncologist closely monitor the effects of the platinum-containing compound treatment and, if necessary, prematurely terminate the treatment. Premature termination removes an important treatment from the armamentarium of the oncologist for treating cancers, e.g., head and neck, lung, cervix, bladder, testicular, ovary and endometrial tumors.

The inventor has discovered that T cells, such as CD4⁺ T cells and CD8⁺ T cells, mediate the toxic effects of platinum-containing compounds on the kidney. In the absence of T cells, the toxic effects do not occur. Therefore, treatment of a patient receiving or about to receive platinum-containing compound in a manner which depletes T cells or modulates their activity, permits platinum-containing compounds to be used safely without premature termination.

Drugs for which the present invention applies include the platinum-containing compounds. Food and Drug Administration-approved members of this family include cisplatin, carboplatin, and oxaliplatin. These are used for treating a broad variety of cancers. Other such compounds include spiroplatin, iproplatin, JM216, AMD473, and BBR3464. Other drugs which cause nephrotoxicity can also be used in conjunction with the kidney-saving treatments of the present invention. These drugs include both injectable and non-injectable drugs, chemotherapy drugs, antibiotics and contrast dyes. Specific nephrotoxic drugs with which the present methods may be used include: mitomycin C, bisphosphonates, methotrexate, streptozotocin, nitrosoureas, cyclosporine, amphotericin, bifosfamide, cyclophosphamide, interferon-alpha, aminoglycoside antibiotics, X-ray contrast dyes, gemcytobine, and deoxycoformycin. T cells can be depleted or modulated according to the present invention for prevention, amelioration, and alleviation of kidney damage due to any of these nephrotoxic drugs or agents.

Antibodies can be used to deplete T cells in the patient under treatment. Antibodies may be directed to T cells generically, to groups of T cells, or to particular types of T cells. Antibodies to T cells generically include Thymoglobulin™ (Genzyme, Cambridge, Mass.). Thymoglobulin is a polyclonal antibody that suppresses certain types of immune cells responsible for acute organ rejection in transplant patients. Thymoglobulin is a mixture of antibodies which bind to various cell surface antigens. Other suitable antibodies for this purpose are those which are anti-CD4, anti-CD8, anti-CD28, anti-CD3, anti-CD52 (e.g., alemtuzumab (Campath®; Genzyme, Cambridge, Mass.)), anti-CD154, anti-ICOS receptor, anti-PD1, and mAb Hyb-241 (Hybritech). The latter is a mouse monoclonal antibody which detects an extracellular epitope of P-glycoprotein. Antibodies to ligands for cell surface markers may also be used to deplete antibodies. Such ligands include T cell co-stimulatory factors B7h (the ICOS ligand), PD-L1 and PD-L2. Any antibodies, whether polyclonal or monoclonal, whether mouse, rabbit, goat, human, humanized, chimeric, etc., can be used which bind to and deplete T cells. The T cell markers and antibodies mentioned here are well known in the art. One particular combination which can be used to good effect is a mixture of antibodies to CD4, CD8, and Thy 1.2 (CD90) antigens. Other combinations of such antibodies can be used as well. For example, anti-CD4 antibodies can be used with any antibody directed against one or more of CD8, CD28, anti-CD52, anti-ICOS receptor, anti-PD1, CD3 (e.g., OKT3; Orthoclone), CD154. Treatment with a combination of antibodies may be done sequentially or at one time as a cocktail or mixture. The antibodies in a combination can be raised against the same or different antigens. Typical doses of antibodies are 0.1-30 mg/kg/day, 0.5-1.5 mg/kg/day, and 1-2 mg/kg/day, 3-10 mg/kg/day. Potency of different antibodies will differ, and these doses are meant to be exemplary only.

Drugs can be used according to the present invention for downwardly modulating the activity of T cells in the patient under treatment. Such drugs include mycophenolate mofetil (CellCept™; Roche), IL-10, TGF-beta, CD152, CTLA-4-Ig (Abatacept™), Tamoxifen (e.g., Nolvadex D®, Soltamox®, Tamofen®), and TJU103 (N-(3-Indolylmethylene)-isonicotinic hydrazone; Alexis). Other drugs, including corticosteroids and other immunosuppressives that dampen T cell function, as are known in the art can be used as well. Exemplary corticosteroids include betamethasone, budesonide, cortisone, dexamethasone, hydrocortisone, methylprednisolone, prednisolone, prednisone, triamcinolone, betamethasone. These drugs can be used alone or in combinations with each other or with other drugs.

Prevention and treatment as used herein refer to methods that are used either before or after, respectively, the onset of kidney toxicity and/or failure. The methods need not be 100% effective to qualify as either prevention or treatment. Methods that reduce the rate of occurrence in a population or which reduce the severity of symptoms qualify as prevention or treatment. According to certain embodiments of the invention, the level of CD4⁺ T cells in the patient's peripheral blood after treatment for depletion is reduced to less than 50%, 40%, 30%, 20%, 10%, or 5% of the pretreatment or untreated level. According to certain other embodiments of the invention, the level of IFN-γ in the patient's peripheral blood is reduced after treatment for depletion or modulation is reduced to less than 50%, 45%, 40%, 35%, 20%, or 10% of the pretreatment or untreated or unmodulated level.

Antibodies, small organic molecules, or other agents for depletion of T cells, such as CD4⁺ T cells, or for modulation of their activity can be administered by any means known in the art. Typically such agents will be injected or infused intravenously, although other routes of administration are possible. Other routes include, without limitation, intraperitoneal, intramuscular, transdermal, subcutaneous, per os. Direct administration to the spleen, thymus, or lymph nodes, sites of T cell production, maturation, or concentration, can also be used. Dosage of such agents can be those that are recommended by the manufacturer for other uses of these agents.

Kits can be formulated for treatments according to the present invention. Such kits comprise at least two components. The components are packaged within a single divided or undivided container. Instructions for using the components may be present as printed matter, on an electronic medium, or as a reference to an internet site. The components include a drug or agent which causes undesirable nephrotoxicity, as discussed above, and an agent for treating or preventing the nephrotoxicity, also as discussed above. According to one embodiment, the agent for treating or preventing nephrotoxicity is a platinum-containing compound. More than one nephrotoxicity-causing drug or agent and/or more than one nephrotoxicity-treating or -preventing agent may be included in the kit, either as mixtures or as separate components.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Materials and Methods

Animals

All animal study protocols have been reviewed and approved by the Animal Care and Use Committee of Johns Hopkins University (IACUC), and all experiments were conducted according to NIH guidelines. T cell deficient athymic male mice (B6.Cg-Foxn1^{nu}, nu/nu) and their C57BL/6 wild type male littermates (6-8 wks, weighing 20-25 g) were purchased from The Jackson Laboratory (Bar Harbor, Me., USA). The two main defects of T cell deficient mice homozygous for the nu/nu spontaneous mutation (Foxn/nu, formerly Hfh11nu) are the abnormal hair growth and defective development of the thymus. Consequently, homozygous nu/nu mice lack T cells and cell-mediated immunity. Genetically matched wild type male littermates were used as controls and as donors of T cells adoptive transfer. CD4-deficient mice (B6.129S2-Cd4tm1Mak), CD8-deficient mice (B6.129S2-Cd8atm1Mak) and their wild type littermates were also purchased from The Jackson Laboratory. Mice were held under pathogen-free conditions in JHMI animal facility with air conditioning, 14 hr/10 hr of light and dark cycle and were free access to food and water during the experiments.

Cisplatin Administration and Tissue Collection

Cisplatin (cis-diammineplatinum II dichloride, Sigma-Aldrich, St.Louis, Mo.) was dissolved in 0.9% of saline at a concentration of 1 mg/ml. Mice were given a single i.p. injection either with cisplatin (40 mg/kg body weight) or with equal volume of saline. This dose was chosen based on our preliminary studies that lower doses did not give a consistent and significant renal dysfunction and tubular injury, but a dose of 40 mg/kg produced a predictable combination of survivability and acute renal failure from as early as 24 hr and reached a peak at 72 hr after cisplatin administration in C57BL6 wild type mice. For kidney T cell immunohistochemical staining, three wild type mice in each group were sacrificed at 1 hr, 6 hr, 12 hr, 24 hr or 72 hr after cisplatin injection. All nu/nu or knockout mice and their littermates were sacrificed at 24 hr or 72 hr after the cisplatin administration for histology or for kidney cytokine array. All collected mice kidneys were either fixed in 10% buffered formalin for histology/immunohistochemistry, or snap frozen with liquid nitrogen for tissue cytokine array.

Assessment of Renal Function

Blood samples were obtained from mice tails prior to (0 hr) and at 24 hr, 48 hr and 72 hr after cisplatin injection. Serum creatinine was measured as a marker of renal dysfunction by a Roche Cobas Fara automated system (Roche, Nutley, N.J., USA) using a Creatinine 557 kit (Sigma Diagnostics, St. Louis, Mo., USA).

Histological Examination

Formalin-fixed paraffin-embedded sections of mice kidneys tissues were cut and stained with hematoxylin and eosin (H&E). Renal tubular injury was assessed using a semi-quantitative scale. A pathologist blinded to the experiments scored the degrees of tubular injury. The magnitude of tubular epithelial cell loss, necrosis, intra-tubular debris and tubular cast formation was scored into six levels based on the percentage of affected tubules in a high power field (HPF) under light microscope. (0: none; 0.5: <10%; 1: 10 to 25%; 2: 25 to 50%; 3: 50 to 75%; 4: >75%).

T Cell Adoptive Transfer

One group of nu/nu mice (n=5) received adoptive transfer of T cells from their littermate wild type mice. Briefly, spleens collected from the normal C57BL/6 wild type littermate mice were minced on a nylon mesh and filtered through a cell strainer (70 μm). The obtained cell suspension was centrifuged to obtain splenocytes pellet. The red blood cells were removed by using a RBC lysis buffer (eBioscience, San Diego, Calif.). T cells were enriched by a nylon wool column chromatography (R&D System, Minneapolis, Minn.) according the manufacture's instructions. After enrichment treatment, the purity of T cell suspension was greater than 90%. Approximately 3×10⁶ enriched T cells were injected i.p. into each nu/nu mouse three weeks before cisplatin administration. This interval was chosen based on our preliminary studies which showed earlier time points had less efficient reconstitution. (14)

FACS analysis of Splenic T Cell Reconstitution

To confirm T cell reconstitution in nu/nu mice, the spleens were collected from nu/nu mice with or without T cells transfer and from their wild type littermates upon sacrifice at 72 hr post cisplatin. The splenocytes were isolated and analyzed for a population of CD3+ cells (pan T cells) by flow cytometry. Briefly, the splenocytes were isolated from collected mice spleens. After the red blood cells were removed, the remaining cells were blocked with an Fcγ III/II receptor and directly stained with a FITC conjugated anti-mouse CD3 (17A2) monoclonal antibody (BD PharMingen, San Diego Calif.) for 20 minutes at room temperature. The stained cells were then fixed in 1% of formalin solution, and then analyzed by FACScaliber using the Cell Quest software V3.3 (Becton Dickinson Immunocytometry Systems, San Jose, Calif.). The CD3 positive population was expressed as a percentage of all gated lymphocytes.

Immunohistochemical Staining of T Cells

In order to evaluate T cell infiltrating into post cisplatin kidneys as a potential mechanism of action, immunohistochemical staining for T cell was performed on formalin-fixed kidney tissue. Briefly, after deparaffinization and rehydration, kidney sections were immersed in 3% hydrogen peroxide methanol for 5 minutes to block endogenous peroxidase. For antigen retrieval, slides were pressure-cooked in Antigen-decloaker solution (Biocare Medical, Walnut creek, Calif.) for 3 minutes. After treatments with normal goat serum (1:100) and two drops of avidin D (100 mg/ml PBS), a polyclonal rabbit anti human/mouse CD3 antibody was added at a 1:200 dilution (Calbiochem®, San Diego, Calif.) for overnight at 4° C., and followed by a incubation with a biotinylated goat anti rabbit IgG for 35 minutes and with Streptavidin Peroxidase (Biogenex, San Ramon, Calif.) for 45 minutes. Finally the kidney sections were exposed with Romulin AEC Chromogen (Biocare Medical, Walnut Creek, Calif.) to visualize the immuno-complex and counterstained with hematoxylin. All cisplatin kidney sections were examined by a pathologist and a nephrologist in a blinded fashion, and CD3 positive cells were counted in at least 10 cortico-medulary fields.

Kidney Myeloperoxidase Assay

Renal myeloperoxidase (MPO) activity was measured as described by Laight et al. (18) in the nu/nu mice and wild type mice at 72 hr after receiving cisplatin to semi-quantify neutrophils and macrophages infiltration. Briefly, kidney tissue was homogenized in a solution containing 0.5% (wt/vol) hexadecyltrimethylammonium bromide dissolved in 50 mM potassium phosphate buffer (pH 6.0) and centrifuged for 30 min at 20,000 g at 4° C. Samples were incubated in a water bath at 60° C. for 2 hr and then centrifuged at 4,000 g for 12 min. The collected supernatant (40 μl) in each sample was incubated with 160 μl of a reaction solution containing 1.6 mM tetramethylbenzidine and 3 mM H₂O₂ diluted in 80 mM phosphate buffer (pH 5.4) in a 96-well microplate. The rate of change in absorbance at 630 nm over 5 min was measured spectrophotometrically. MPO activity was expressed as absorbance changed per minute per 100 mg of wet tissue.

Kidney Cytokine Protein Array

In order to examine pro-inflammatory molecules generated by cisplatin, protein levels of IL-1β, IFN-γ, TNF-α and KC was measured in the mice kidney by using Bio-Rad Bio-Plex™ multiple cytokines array technique (Bio-Rad Laboratories Inc., Hercules, Calif.) described elsewhere in depth (19) with a simplified small template containing only above four cytokine/chemokines. Briefly, snap frozen kidney tissues were homogenized in a kit-attached cell-lysis buffer, and the homogenates were centrifuged at 12,000 rpm for 15 min at 4° C. Total protein concentration in each supernatant was determined by using a Bio-Rad Protein Assay I(it, and was adjusted to 500 μg/ml with the cell lysis buffer. Each sample was first incubated with a mixture of all types of micro-beads for 90 min at room temperature (RT) followed by an incubation with biotinylated detection antibodies for 30 min and then with a strepavidin-coupled phycoerythrin for 10 min (RT). Finally, the samples were subjected to a flow cytometric system. All acquired data was analyzed using Bio-Plex Manager™ 3.0 software (Bio-Rad) and corrected by total protein concentration (pg/mg protein).

Statistics

Data are expressed as mean±standard error of mean (SEM) and are compared by unpaired, two-tailed Student t tests for single comparison or by ANOVA Post Hoc test for multiple comparison. Kaplan-Meier analysis was used for mice survival analyses. Statistical significance of difference was defined when the P-value was less than 0.05.

### EXAMPLE 2

### T Cell Deficient Mice Survival After Cisplatin

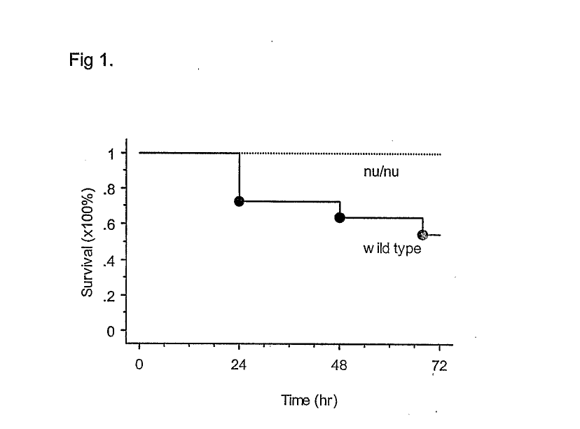

T cell deficient (nu/nu) mice and their C57BL6 wild type littermate mice were received a single i.p. injection of cisplatin at the dose of 40 mg/kg. By 72 hr after injection, 6/14 of the wild type mice were dead (58% survival). Meanwhile, 0/12 of nu/nu mice died, i.e., all of them were alive 72 hrs after cisplatin, (100% of survival; FIG. 1).

### EXAMPLE 3

### T Cell Deficient Mice Markedly Protected from Cisplatin-Induced Renal Dysfunction

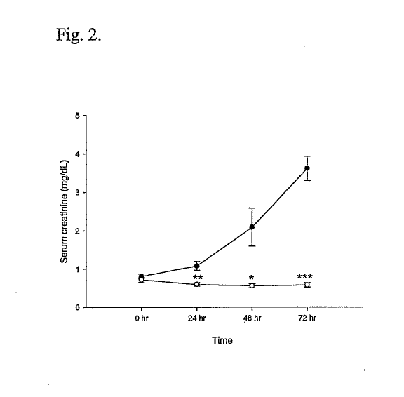

Cisplatin administration led to the development of acute renal failure with a rise in serum creatinine from 0.7 mg/dL (base line) to 3.6 mg/dL by 72 hr post injection in the wild type mice. In contrast, the nu/nu mice received cisplatin had significant attenuation in serum creatinine elevation at 24 hr (1.05±0.11 vs. 0.60±0.05, P<0.02), 48 hr (2.09±0.49 vs. 0.56±0.05, P<0.05) and at 72 hr (3.61±0.32 vs. 0.58±0.06, P<0.0001) (FIG. 2) when compared with wild type mice.

### EXAMPLE 4

### T Cell Deficiency Protects Mice from Renal Tubular Injury Induced by Cisplatin

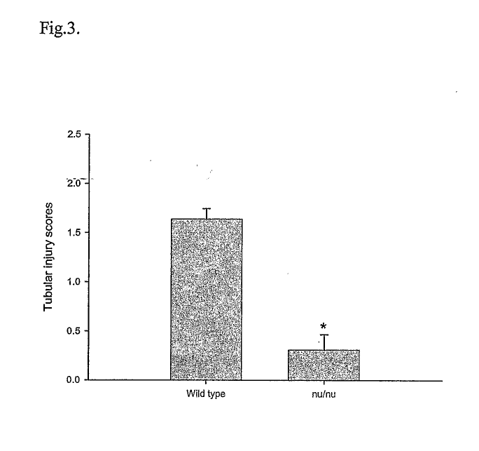

At 72 hr after cisplatin administration, the wild type mice developed extensive renal tubular injury. However, the nu/nu mice had significant less tubular injury. (Injury scores: 1.44±0.15 vs. 0.22±0.08, P<0.0001. FIG. 3)

### EXAMPLE 5

### T Cell Adoptive Transfer Reconstituted Kidney Susceptibility to Cisplatin Toxicity

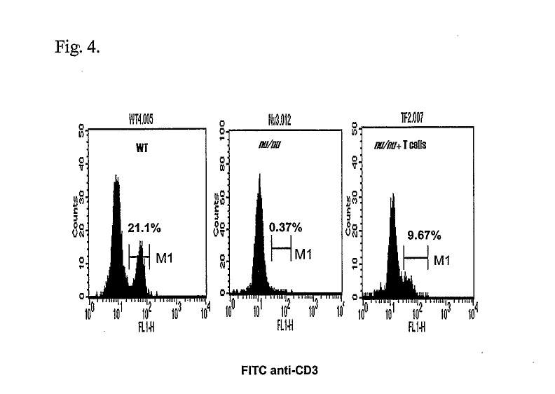

To determine if T cell deficiency in nu/nu mice was indeed the protective factor in cisplatin-induced renal injuries, we transferred 3×10⁶ of purified splenic T cells (purity: greater than 90%) from normal wild type mice into each of five nu/nu mice. The success of T cells reconstitution was confirmed by FACS analysis with CD3 staining. The mean population of T cells in wild type mice spleen was 7.7% of total splenocytes. Meanwhile, nu/nu mice had minimal (0.4%) splenic T cells. After a transfer, The splenic T cells in nu/nu mice were reconstituted to 2.4% (FIG. 4).

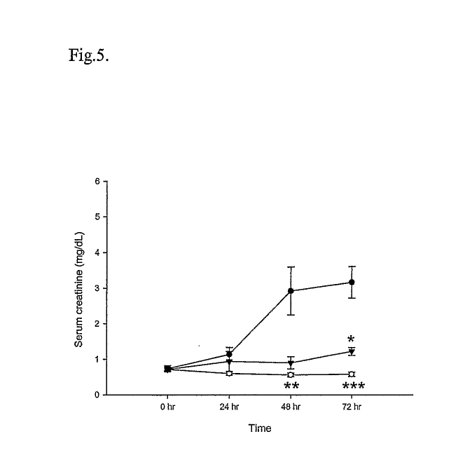

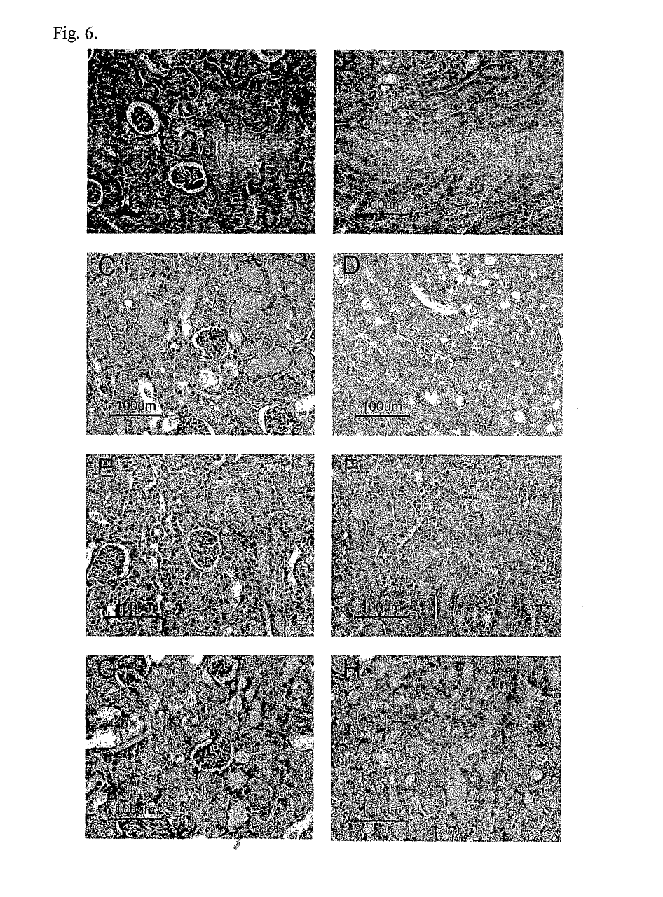

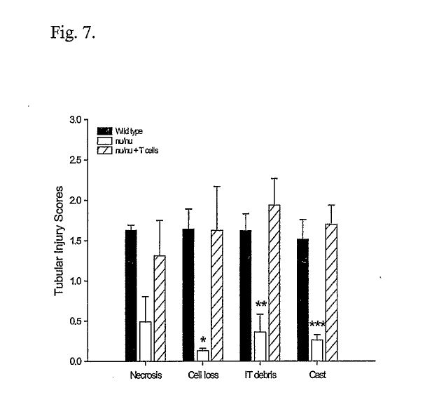

T cell transfers led to a significant enhancement of renal dysfunction in nu/nu mice. There was a significant rise in serum creatinine in the nu/nu mice transferred with T cells when compared to the nu/nu mice alone (without transfer) at 72 hr after cisplatin administration. (0.58±0.06 vs. 1.23±0.11, P<0.04. FIG. 5). The nu/nu mice with T cells transfer developed significant renal tubular injury (FIG. 6), confirmed by a semi-quantitative scoring on tubular injuries in a blinded fashion (FIG. 7). Also, when compared to nu/nu mice alone, the nu/nu mice with T cells transfer had a worse survival (80% vs. 100%, figure not shown).

### EXAMPLE 6

### T Cells Infiltrated Early Into Post Cisplatin Mice Kidneys

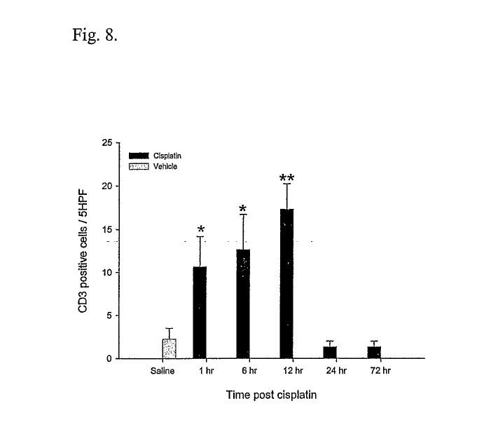

To investigate if T cells track into post cisplatin kidney at early time, three wild type mice in each of five groups were treated with a single dose of cisplatin and were sacrificed at 1 hr, 6 hr, 12 hr and 24 hr after cisplatin injection. Kidney tissues were stained with anti-CD3 antibody, a pan T cell surface marker, by immunohistochemistry. A markedly increased CD3 positive cells were detected in the mice kidney as early as at 1 hr post cisplatin, this increase of T cells reached a peak at 12 hr, and then declined by 24 hr. (FIG. 8 *, P<0.004; **, P<0.0002)

### EXAMPLE 7

### T Cell Deficiency Attenuates Increased Renal Myeloperoxidase Activity Late After Cisplatin

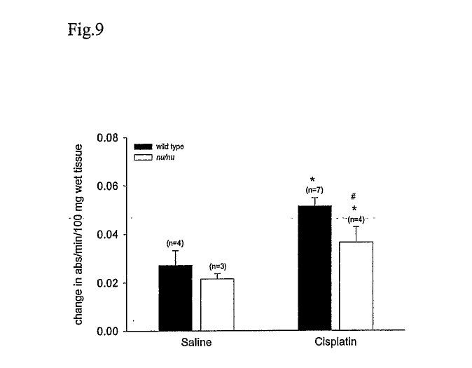

In order to assess renal phagocyte infiltration after cisplatin treatment, MPO activity in post cisplatin kidneys was measured in nuinu mice and wild type mice treated either with cisplatin or with saline as negative controls. Compared to their individual saline controls, both wild type and nu/nu mice had significant increase in renal MPO activities at 72 hr after receiving cisplatin. However, this increase was significantly blunted in the nu/nu mice when compared with the wild type mice. (FIG. 9)

### EXAMPLE 8

### Both CD4 Deficient Mice and CD8 Deficient Mice were Protected from Cisplatin Induced Mortality and Renal Dysfunction

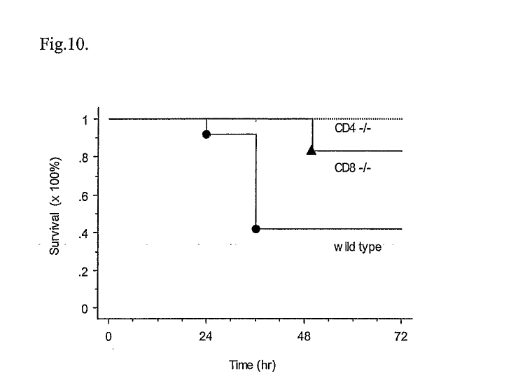

To dissect the individual roles of the CD4 and CD8 T cell subsets on the outcome of cisplatin induced renal injury, we evaluated the effects of cisplatin on acute renal failure in the mice that were deficient in either CD4 or CD8 T cell. These deficient mice as well as their littermates received a single dose (40 mg/kg, i.p.) of cisplatin and were followed up to 72 hr. By 72 hr after cisplatin treatment, 7/12 wild type mice died, meanwhile only 1/6 CD8 deficient mice died, and 0/6 CD4 deficient mice died. (FIG. 10). Wild type mice developed a significant rise in serum creatinine, while both CD4 deficient mice and CD8 deficient mice showed much milder renal dysfunction at 24 hr post cisplatin treatment. (SCr, mg/dL: WT vs. CD4−/− vs. CD8−/−, 1.74±0.25 vs. 0.50±0.07 vs. 0.67±0.08, P<0.001 or <0.004, figure not shown)

### EXAMPLE 9

### T Cell Deficiency Attenuates Renal Cytokines/Chemokines Protein Production After Cisplatin

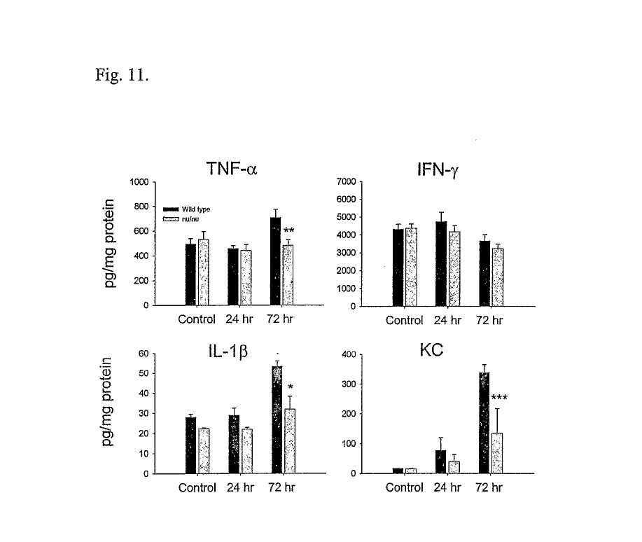

To determine potential soluble mediators of T cell in cisplatin induced nephrotoxicity, we measured protein levels of IL-β, KC, IFN-γ and TNF-α in the mouse kidney at 24 hr and 72 hr after cisplatin injection. Compared to the saline controls, cisplatin-treated wild type mice had increased levels of renal IL-1β, KC and TNF-α (all as pg/mg protein) at 72 hr after the injection (IL-1β: 27.91±1.62 vs. 53.43±2.61, P=0.0005; KC: 16.87±0.38 vs. 338.30±27.04, P=0.0004; TNF-α: 495.21±44.05 vs. 707.40±66.28, P<0.02). However, compared to the wild type mice, the nu/nu mice treated with cisplatin had a reduced expression of these pro-inflammatory molecules in the kidney. There was no increase in IFN-γ protein levels both in the wild type and the nu/nu mice at any time point after cisplatin administration. (FIG. 11)

### EXAMPLE 10

### Discussion

Nephrotoxicity is a major limitation for administering adequate doses of the metal chemotherapeutic agent cisplatin. Most studies support a role for apoptosis/necrosis and reactive oxygen species in the pathogenesis of cisplatin-induced renal injury (3;8). Recently, an inflammatory basis for cisplatin toxicity has been demonstrated with a role for ICAM-1, TNF-α and other pro-inflammatory molecules (10;11;20). The above data demonstrate for the first time that T cells directly mediate the pathogenesis of cisplatin-induced acute nephrotoxicity. The marked functional and structural protection seen in the T cell deficient nu/nu mice corresponded with a survival advantage.

In order to confirm that T cell deficiency in nu/nu mice is directly related to the protective effect, T cell adoptive transfers were performed on some nu/nu mice, which significantly restored both structural and functional injury to cisplatin. T cells infiltrated very early into wild type mouse kidneys within hours after cisplatin treatment, but these T cells were not present after 24 hrs, demonstrating a potential role for early T cells trafficking in this toxic process. Both CD4 and CD8 T cells appear to play an important role in cisplatin toxicity given the improved survival and renal protection seen in CD4 or CD8 individual knock out mice. Infiltration of neutrophils and macrophages into kidney represented by renal MPO activities late after cisplatin was significantly attenuated by T cell deficiency, indicating a potential effector mechanism of T lymphocyte.

Proinflammatory molecules were also measured in the kidney in this study to explore other potential effector mechanisms. After cisplatin treatment, the protein levels of TNF-α, KC and IL-1β were increased in the wild type mouse kidneys, and these increases were blunted in the nu/nu mice kidneys.

The marked degree of protection from renal functional decline and structural injury in the T cell deficient nu/nu mouse strain was unexpected. Given that the T cell deficient nu/nu mice could have other abnormalities besides T cell deficiency leading to resistance to cisplatin toxicity, we then studied the effects of adoptive transfer of wild type T cells into nu/nu mice on cisplatin toxicity. We found significant worsening in kidney function and tubular injury in T cell transferred nu/nu mice compared to the nu/nu mice alone. Approximately 9% of T cells were found in the transferred nu/nu mice spleens, compared to 21% in wild type. Thus, even low numbers of T cells are sufficient to mediate cisplatin nephrotoxicity(14).

We then began to explore the potential mechanisms by which T cells could mediate cisplatin-induced nephrotoxicity. T cell trafficking into a target organ is an important basis for T cell-mediated injury in many diseases such as transplant rejection (22). Using a polyclonal antibody specific for detecting pan T cells in mouse tissue, we found a significant increase in T cell infiltration into post cisplatin mouse kidney within hours of cisplatin administration, that decreased by 24 and 72 hrs. This early trafficking could underlie the strong effects of T cells in this nephrotoxic model. However, there are examples in other inflammatory diseases such as in experimental asthma where one can dissociate T cell trafficking into involved tissue even when there is likely a T cell-mediated functional effect(23).

We found that CD4 deficiency conferred a marked renal function and mortality protection from cisplatin. This may be mediated through the production of deleterious Th1 polarized cytokines from CD4 cells (24). We also unexpectedly found a significant protection afforded by CD8 deficiency, though this was not as protective as CD4 deficiency. Thus, both CD4 and CD8 T cells appear to mediate cisplatin induced nephrotoxicity. Interestingly, in a murine model of adriamycin induced nephirotoxicity, CD4 T cells, which are traditionally considered as a regulator, have been shown to improve outcome while CD8 T cells, a scavenger, promoted renal injury. (25;26)

Even though early recruitment of T cells was observed, their small numbers suggested that other potent effector mechanisms were in play. We therefore measured infiltrating phagocytes (neutrophils and macrophages) using myeloperoxidase (MPO) activity assay. Kidney MPO levels were increased in both WT and nu/nu mice after cisplatin, however, this increase of MPO activities in post cisplatin kidney was significantly reduced in nu/nu mice. Thus, T cell mediated phagocyte infiltration is a potential mode of action, and is consistent with recent publications on the mechanisms of T cells in ischemic tissue injury (17;27). Given that recent data has demonstrated a significant role for TNF-α in cisplatin nephrotoxicity, we measured other cytokine/chemokines protein besides TNF-α in both cisplatin treated nu/nu mice and wild type mice kidneys as potential mediators of the T cell's role in cisplatin nephrotoxicity. We found an increase in TNF-α, IL-1β and KC at the time of the rise in serum creatinine and tubular injury in wild type mice. However, these increases were blunted in the nu/nu mice. Thus, these molecules may be potential effectors of the T cell mediated cisplatin toxicity. Alternatively, these could be associated with the decreased injury rather than cause and effect.

An improved understanding of the pathophysiology of cisplatin nephrotoxicity should lead to improved preventive and therapeutic strategies. Peroxisome proliferator-activated receptor-alpha (PPAR alpha) ligands can be used also to prevent cisplatin nephrotoxicity (28). This compound has been shown to have profound inhibitory effect on T cell function by impaired production of TNF-α (29).

### REFERENCES

The disclosure of each reference cited is expressly incorporated herein.
1. Schrier R W: Cancer therapy and renal injury. J. Clin. Invest 110:743-745, 2002
2. Ries F, Klastersky J: Nephrotoxicity induced by cancer chemotherapy with special emphasis on cisplatin toxicity. Am. J. Kidney Dis 8:368-379, 1986
3. Matsushima H, Yonemura K, Ohishi K, Hishida A: The role of oxygen free radicals in cisplatin-induced acute renal failure in rats. J. Lab Clin. Med 131:518-526, 1998
4. Kaushal G P, Kaushal V, Hong X, Shah S V: Role and regulation of activation of caspases in cisplatin-induced injury to renal tubular epithelial cells. Kidney Int 60:1726-1736, 2001
5. Leibbrandt M E, Wolfgang G H, Metz A L, Ozobia A A, Haskins J R: Critical subcellular targets of cisplatin and related platinum analogs in rat renal proximal tubule cells. Kidney Int 48:761-770, 1995
6. Megyesi J, Safirstein R L, Price P M: Induction of p21WAF1/CIP1/SDI1 in kidney tubule cells affects the course of cisplatin-induced acute renal failure. J. Clin. Invest 101:777-782, 1998
7. Sugiyama S, Hayakawa M, Kato T, Hanaki Y, Shimizu K, Ozawa T: Adverse effects of anti-tumor drug, cisplatin, on rat kidney mitochondria: disturbances in glutathione peroxidase activity. Biochem. Biophys. Res. Commun 159:1121-1127, 1989
8. Lieberthal W, Triaca V, Levine J: Mechanisms of death induced by cisplatin in proximal tubular epithelial cells: apoptosis vs. necrosis. Am. J. Physiol 270:F700-F708, 1996
9. Okuda M, Masaki K, Fukatsu S, Hashimoto Y, Inui K: Role of apoptosis in cisplatin-induced toxicity in the renal epithelial cell line LLC-PK1. Implication of the functions of apical membranes. Biochem. Pharmacol 59:195-201, 2000
10. Ramesh G, Reeves W B: TNF-alpha mediates chemokine and cytokine expression and renal injury in cisplatin nephrotoxicity. J. Clin. Invest 110:835-842, 2002
11. Ramesh G, Reeves W B: TNFR2-mediated apoptosis and necrosis in cisplatin-induced acute renal failure. Am. J. Physiol Renal Physiol 285:F610-F618, 2003
12. Ramesh G, Reeves W B: Salicylate reduces cisplatin nephrotoxicity by inhibition of tumor necrosis factor-alpha. Kidney Int 65:490-499, 2004
13. Kim Y K, Choi T R, Kwon C H, Kim J H, Woo J S, Jung J S: Beneficial effect of pentoxifylline on cisplatin-induced acute renal failure in rabbits. Ren Fail 25:909-922, 2003
14. Bume M J, Daniels F, El Ghandour A, Mauiyyedi S, Colvin R B, O'Donnell M P, Rabb H: Identification of the CD4(+) T cell as a major pathogenic factor in ischemic acute renal failure. J. Clin. Invest 108:1283-1290, 2001
15. Zwacka R M, Zhang Y, Halldorson J, Schlossberg H, Dudus L, Engelhardt J F: CD4(+) T-lymphocytes mediate ischemia/reperfusion-induced inflammatory responses in mouse liver. J. Clin. Invest 100:279-289, 1997
16. de Perrot M, Young K, Imai Y, Liu M, Waddell T K, Fischer S, Zhang L, Keshavjee S: Recipient T cells mediate reperfusion injury after lung transplantation in the rat. J. Immunol 171:4995-5002, 2003
17. Shigematsu T, Wolf R E, Granger D N: T-lymphocytes modulate the microvascular and inflammatory responses to intestinal ischemia-reperfusion. Microcirculation 9:99-109, 2002
18. Laight D W, Lad N, Woodward B, Waterfall J F: Assessment of Myeloperoxidase Activity in Renal Tissue After Ischaemia/Reperfusion. European Journal of Pharmacology-Environmental Toxicology and Pharmacology Section 292:81-88, 1994
19. Hensley K, Fedynyshyn J, Ferrell S, Floyd R A, Gordon B, Grammas P, Hamdheydari L, Mhatre M, Mou S, Pye Q N, Stewart C, West M, West S, Williamson K S: Message and protein-level elevation of tumor necrosis factor alpha (TNF alpha) and TNF alpha-modulating cytokines in spinal cords of the G93A-SOD1 mouse model for amyotrophic lateral sclerosis. Neurobiol. Dis 14:74-80, 2003
20. Kelly K J, Meehan S M, Colvin R B, Williams W W, Bonventre J V: Protection from toxicant-mediated renal injury in the rat with anti-CD54 antibody. Kidney Int 56:922-931, 1999
21. Rabb H, Daniels F, O'Donnell M, Haq M, Saba S R, Keane W, Tang W W: Pathophysiological role of T lymphocytes in renal ischemia-reperfusion injury in mice. Am. J. Physiol Renal Physiol 279:F525-F531, 2000
22. Jones T R, Shirasugi N, Adams A B, Pearson T C, Larsen C P: Intravital microscopy identifies selectins that regulate T cell traffic into allografts. J. Clin. Invest 112:1714-1723, 2003
23. Rabb H, Martin J G: An emerging paradigm shift on the role of leukocyte adhesion molecules. J. Clin. Invest 100:2937-2938, 1997
24. Yokota N, Bume-Taney M, Racusen L, Rabb H: Contrasting roles for STAT4 and STAT6 signal transduction pathways in murine renal ischemia-reperfusion injury. Am. J. Physiol Renal Physiol 285:F319-F325, 2003
25. Wang Y, Wang Y, Feng X, Bao S, Yi S, Kairaitis L, Tay Y C, Rangan G K, Harris D C: Depletion of CD4(+) T cells aggravates glomerular and interstitial injury in murine adriamycin nephropathy. Kidney Int 59:975-984, 2001
26. Wang Y, Wang Y P, Tay Y C, Harris D C: Role of CD8(+) cells in the progression of murine adriamycin nephropathy. Kidney Int 59:941-949, 2001
27. Singbartl K, Bockhom S G, Zarbock A, Schmolke M, Van Aken H: T cells modulate neutrophil-dependent acute renal failure during endotoxemia: critical role for CD28. J. Am. Soc. Nephrol 16:720-728, 2005
28. Li S, Wu P, Yarlagadda P, Vadjunec N M, Proia A D, Harris R A, Portilla D: PPAR alpha ligand protects during cisplatin-induced acute renal failure by preventing inhibition of renal FAO and PDC activity. Am. J. Physiol Renal Physiol 286:F572-F580, 2004
29. Cunard R, DiCampli D, Archer D C, Stevenson J L, Ricote M, Glass C K, Kelly C J: WY14,643, a PPAR alpha ligand, has profound effects on immune responses in vivo. J. Immunol 169:6806-6812, 2002

I claim:

1. A method to prevent platinum-containing compound-induced kidney toxicity in a patient, comprising:
depleting T cells in the patient prior to a planned administration or concomitant with administration of a platinum-containing compound.

2. A method to treat platinum-containing compound-induced kidney toxicity in a patient in need thereof, comprising:
depleting T cells in a patient that has been treated with a platinum-containing compound.

3. The method of claim 1 or 2 wherein the T cells are CD4⁺ T cells.

4. The method of claim 1 or 2 wherein the T cells are CD4⁺ CD8⁺ T cells

5. The method of claim 3 wherein the CD4⁺ T cells are depleted to a level which is less than 50% of the untreated level in peripheral blood.

6. The method of claim 3 wherein the CD4⁺ T cells are depleted to a level which is less than 40% of the untreated level in peripheral blood.

7. The method of claim 3 wherein the CD4⁺ T cells are depleted to a level which is less than 30% of the untreated level in peripheral blood.

8. The method of claim 3 wherein the CD4⁺ T cells are depleted to a level which is less than 20% of the untreated level in peripheral blood.

9. The method of claim 3 wherein the CD4⁺ T cells are depleted to a level which is less than 10% of the untreated level in peripheral blood.

10. The method of claim 3 wherein the CD4⁺ T cells are depleted to a level which is less than 5% of the untreated level in peripheral blood.

11. The method of claim 1 or 2 wherein the step of depleting is performed using antibodies.

12. The method of claim 11 wherein the antibodies are selected from the group consisting of: anti-CD4, anti-CD8, anti-CD28, anti-CD3, anti-CD52, anti-ICOS receptor, anti-PD1, anti-CD154, and mAb Hyb-241.

13. The method of claim 11 wherein the antibodies specifically bind to a T cell co-stimulatory molecule.

14. The method of claim 1 or 2 wherein the step of depleting is performed using a cocktail of antibodies raised against different antigens.

15. The method of claim 1 or 2 wherein the step of depleting is performed using antibodies to CD4, CD8, and Thy 1.2 antigens.

16. The method of claim 1 or 2 wherein the patient has a tumor.

17. The method of claim 1 or 2 wherein the platinum containing compound is cisplatin.

18. The method of claim 1 or 2 wherein the platinum containing compound is carboplatin.

19. The method of claim 1 or 2 wherein the platinum containing compound is oxaliplatin.

20. A method to prevent platinum-containing compound-induced kidney toxicity in a patient, comprising:
modulating T cell activity in a patient such that level of IFN-γ in the patient's peripheral blood is less than 50% of untreated level, wherein the patient is scheduled for treatment with a platinum-containing compound or is treated with a platinum-containing compound concomitantly.

21. A method to treat platinum-containing compound-induced kidney toxicity in a patient, comprising:
modulating T cell activity in a patient such that level of IFN-γ in the patient's peripheral blood is less than 50% of untreated level, wherein the patient has been treated with a platinum-containing compound.

22. The method of claim 20 or 21 wherein the patient has a tumor.

23. The method of claim 20 or 21 wherein the level of IFN-γ is less than 45% of untreated level.

24. The method of claim 20 or 21 wherein the level of IFN-γ is less than 40% of untreated level.

25. The method of claim 20 or 21 wherein the level of IFN-γ is less than 35% of untreated level.

26. The method of claim 20 or 21 wherein an agent selected from the group consisting of IL-10, TGF-beta, CD152, CTLA-4-Ig, Tamoxifen, and TJU103 is administered to the patient.

27. The method of claim 20 or 21 wherein the platinum containing compound is cisplatin.

28. The method of claim 20 or 21 wherein the platinum containing compound is carboplatin.

29. The method of claim 20 or 21 wherein the platinum containing compound is oxaliplatin.

30. A kit for treating cancers, comprising:
a platinum-containing compound; and
an agent selected from the group consisting of: of IL-10, TGF-beta, CD152, CTLA-4-Ig, Tamoxifen, and TJU103;
wherein the platinum-containing compound and the agent are in a single divided or undivided container.

31. A kit for treating cancers, comprising:
a platinum-containing compound; and
an antibody selected from the group consisting of: anti-CD4, anti-CD8, anti-CD28, anti-CD3, anti-CD52, anti-ICOS receptor, anti-PD1, anti-CD154, and mAb Hyb-241.
wherein the platinum-containing compound and the agent are in a single divided or undivided container.

32. The kit of claim 30 or 31 further comprising instructions for administration of the components of the kit to reduce kidney toxicity of the platinum-containing compound.

33. The kit of claim 30 or 31 wherein the platinum-containing compound is cisplatin.

34. The kit of claim 30 or 31 wherein the platinum-containing compound is oxaliplatin.

35. The kit of claim 30 or 31 wherein the platinum-containing compound is carboplatin.
